(19) United States
(12) Patent Application Publication
(10) Pub. No.: US 2025/0035610 A1
(43) Pub. Date: Jan. 30, 2025

(54) CELL POTENTIAL MEASURING DEVICE

(71) Applicant: Sharp Display Technology Corporation, Kameyama City, Mie (JP)

(72) Inventors: Kenichi KITOH, Kameyama City (JP); Fumitoshi YASUO, Kameyama City (JP); Chihiro TACHINO, Kameyama City (JP); Tomoko TERANISHI, Kameyama City (JP); Takeshi HARA, Kameyama City (JP)

(21) Appl. No.: 18/715,903

(22) PCT Filed: Feb. 3, 2022
(86) PCT No.: PCT/JP2022/004307

§ 371 Date: Jun. 3, 2024

(51) Int. Cl. G01N 33/483; C12M 1/34

(52) U.S. Cl. CPC G01N 33/4833; C12M 41/46

(57) ABSTRACT

A cell potential measuring device includes a base plate having insulation properties, first line disposed on the base plate, an insulation layer disposed on the base plate and covering at least a surface of the first line, a first electrode disposed on the insulation layer and being electrically connected to the first line, and a second line disposed on the base plate and a portion of which is disposed below the first electrode via the insulation layer.

### TECHNICAL FIELD

The technology described herein is related to a cell potential measuring device.

### BACKGROUND ART

An action potential of a cell or tissue (simply referred to as a cell hereinafter) has been measured with using a multi-electrode array (MEA) and with in-vitro and non-invasive measurement. For instance, Patent Document 1 discloses a measuring device that includes an insulation base plate and reference electrodes having a relatively low impedance. The reference electrodes are disposed at positions away from microelectrodes by a predefined distance. Patent Document 1 describes that a noise level can be lowered by arranging the reference electrodes as far as possible from the microelectrodes that are used for measuring action potentials of cells.

### PRIOR ART DOCUMENT

### Patent Document

Patent Document 1: Japanese Unexamined Patent Application Publication No. 11-187865

### SUMMARY OF THE INVENTION

### Problem to be Solved by the Invention

If any problems occur in the microelectrodes (measurement electrode) and the connection of the microelectrodes and extending lines (measurement line) extending therefrom or any defect in the extending lines such as disconnection or deformation, a measurement signal may not be obtained or the obtained measurement signals may have variability. Therefore, in the conventional multi-electrode array, it cannot be determined whether the variability of the measurement results is caused by the multi-electrode array or the cell itself.

The technology described herein was made in view of the above circumstances. An object is to provide a cell potential measuring device that can test an electrically conductive state of a measurement electrode and a measurement line.

### Means for Solving the Problem

(1) A cell potential measuring device according to the present technology includes a base plate having insulation properties, a measurement line (a first line) disposed on the base plate, an insulation layer disposed on the base plate and covering at least a surface of the first line, a measurement electrode (a first electrode) disposed on the insulation layer and being electrically connected to the measurement line, and a test line (a second line) disposed on the base plate and a portion of which is disposed below the measurement electrode via the insulation layer. In this description, the term “on” means one is above other via something and also means one is directly on other without having anything therebetween.

(2) According to one aspect of the present technology, in addition to the above configuration (1), the test line may include a test electrode section (a second electrode section) that is opposite the measurement electrode and a line section that extends from the test electrode section, and a distance between the measurement electrode and the test electrode section may be 10 nm or more and 100 μm or less.

(3) According to one aspect of the present technology, in addition to the above configuration (1) or (2), the insulation layer may cover a surface of the test line.

(4) According to one aspect of the present technology, in addition to any one of the above configurations (1) to (3), the test line may be disposed directly on the base plate.

(5) According to one aspect of the present technology, in addition to any one of the above configurations (1) to (3), the insulation layer may include a first insulation layer that is disposed between the base plate and the test line and a second insulation layer that is disposed between the test line and the measurement electrode.

(6) According to one aspect of the present technology, in addition to any one of the above configurations (1) to (3), the test line may include a test electrode section (a second electrode section) that is opposite the measurement electrode and a line section that extends from the test electrode section, and the line section may be disposed directly on the base plate. The insulation layer may include a first insulation layer that is disposed between the base plate and the test electrode section and a second insulation layer that is disposed between the test electrode section and the measurement electrode.

(7) According to one aspect of the present technology, in addition to the above configuration (6), the line section and the test electrode section may be made of different materials.

(8) According to one aspect of the present technology, in addition to any one of the above configurations (1) to (7), the measurement electrode may include transparent electrically conductive material that includes at least one kind selected from the group consisting of tin oxide, zinc oxide, indium zinc oxide, and indium tin oxide.

(9) According to one aspect of the present technology, in addition to any one of the above configurations (1) to (8), at least a portion of the test line and the measurement line may include at least one kind selected from the group consisting of gold, silver, copper, aluminum, tantalum, tungsten, molybdenum, niobium, and titanium.

(10) According to one aspect of the present technology, in addition to any one of the above configurations (1) to (9), the insulation layer may include a first insulation layer at least a portion of which is disposed directly on the base plate and a second insulation layer at least a portion of which is disposed directly under the measurement electrode. Each of the first insulation layer and the second insulation layer may include an overlapping portion that is disposed above the measurement line, and a shield layer having electrical conductivity may be disposed between the overlapping portion of the first insulation layer and the overlapping portion of the second insulation layer.

(11) According to one aspect of the present technology, in addition to any one of the above configurations (1) to (10), the measurement electrode may include a first measurement electrode (a third electrode) and a second measurement electrode (a fourth electrode), and the test line may include a test electrode section (a second electrode section) that is opposite the measurement electrode and a line section that extends from the test electrode section. The test electrode section may include a first test electrode section (a third electrode section) that is opposite the first measurement electrode and second test electrode section (a fourth electrode that is opposite the second measurement electrode, and the first test electrode section and the second test electrode section may be connected to the line section.

(12) According to one aspect of the present technology, in addition to any one of the above configurations (1) to (11), the measurement electrode may include first measurement electrodes (third electrodes) and second measurement electrodes (fourth electrodes), and the measurement line may include first measurement lines (third lines) that are connected to the first measurement electrodes, respectively, and second measurement lines (fourth lines) that are connected to the second measurement electrodes, respectively. The test line may include a test electrode section (a second electrode section) and a line section. The test electrode section may include first test electrode sections (third electrode sections) that are opposite the first measurement electrodes, respectively, via the insulation layer and second test electrode sections (fourth electrode sections) that are opposite the second measurement electrodes, respectively, via the insulation layer, and the line section may include a first line section that is connected to the first test electrode sections and a second line section that is connected to the second electrode sections.

(13) According to one aspect of the present technology, in addition to the above configuration (12), the first measurement electrodes and the second measurement electrodes may be arranged alternately along one direction along a surface of the base plate. The insulation layer may include a first insulation layer and a second insulation layer. The first insulation layer may be disposed between one of the base plate and the first line section and a corresponding one of the first test electrode sections, the second test electrode sections, and the second line section. The second insulation layer may be disposed above the first test electrode sections, the second test electrode sections, and the second line section and below the first measurement electrodes and the second measurement electrodes. The first line section and the first test electrode sections may be connected via trough portions that is through the first insulation layer.

(14) One aspect of the present technology may include, in addition to the above configuration (13), first shield layers and second shield layers. The first shield layers may be disposed above the first measurement lines, respectively, and between the first insulation layer and the second insulation layer. The first shield layers may be connected to the second test electrode sections, respectively, that are adjacent to the first measurement lines, respectively. The second shield layers may be disposed above the second measurement lines, respectively, and between the first insulation layer and the second insulation layer. The second shield layers may be connected to the first test electrode sections, respectively, that are adjacent to the second measurement lines, respectively;

(15) One aspect of the present technology may include, in addition to any one of the above configurations (1) to (13), a wall that projects from the base plate and surrounds the measurement electrode.

(16) One aspect of the present technology may include, in addition to any one of the above configurations (1) to (14), a field effect transistor that is disposed on the base plate and connected to the test line.

(17) One aspect of the present technology may include, in addition to any one of the above configurations (1) to (15), a field effect transistor that is disposed on the base plate and connected to the measurement line.

### Effect of the Invention

According to the technology described herein, an electrically conductive state of a measurement electrode and a measurement line of a cell potential measuring device can be tested.

### BRIEF DESCRIPTION OF THE DRAWINGS

FIG. 1 is a plan view schematically illustrating a portion of a cell potential measuring device according to a first embodiment.

FIG. 2 is a cross-sectional view of the cell potential measuring device along A-A line in FIG. 1.

FIG. 3 is a typical view illustrating the cell potential measuring device of FIG. 1 that measures action potentials of portions of the cells.

FIG. 4 is a graph illustrating an input signal that is input to the cell potential measuring device.

FIG. 5 is a graph illustrating an output signal that is output from the cell potential measuring device in response to the input signal in FIG. 4.

FIG. 6 is a plan view schematically illustrating a cell potential measuring device according to a second embodiment.

FIG. 7 is a cross-sectional view of the cell potential measuring device along B-B line in FIG. 6.

FIG. 8 is a plan view schematically illustrating a cell potential measuring device according to a third embodiment.

FIG. 9 is a cross-sectional view of the cell potential measuring device along C-C line in FIG. 8.

FIG. 10 is a view typically illustrating a cross section of a portion of a cell potential measuring device according to a fourth embodiment and an output signal.

FIG. 11 is a view typically illustrating a cross section of a portion of a cell potential measuring device according to a fourth embodiment and an output signal.

FIG. 12 is a plan view schematically illustrating a cell potential measuring device according to a fifth embodiment.

FIG. 13 is a cross-sectional view of the cell potential measuring device along D-D line in FIG. 12.

FIG. 14 is a cross-sectional view of the cell potential measuring device along E-E line in FIG. 12.

FIG. 15 is a plan view schematically illustrating a cell potential measuring device according to a sixth embodiment.

FIG. 16 is a plan view schematically illustrating a cell potential measuring device according to a seventh embodiment.

FIG. 17 is an enlarged view of a portion of the cell potential measuring device of FIG. 16.

FIG. 18 illustrates a configuration of a test electrode of the cell potential measuring device of FIG. 17.

FIG. 19 is a cross-sectional view along F1-F5 line in FIG. 18

FIG. 20 illustrates an operation of the cell potential measuring device of FIG. 16.

FIG. 21 illustrates another operation of the cell potential measuring device of FIG. 16.

FIG. 22 is a plan view schematically illustrating a cell potential measuring device of a prior art.

FIG. 23 is a cross-sectional view of the cell potential measuring device along G-G line in FIG. 22.

### MODES FOR CARRYING OUT THE INVENTION

### First Embodiment

One preferable embodiment according to the technology described herein will be described below. Technological matters that are other than the matters particularly described herein (for instance, the configuration of the cell potential measuring device described herein) and are necessary for carrying out the present technology (for instance, a cell to be cultivated, cell culture technology, screening of pharmaceutical composition, general matters regarding preparation, and general matters regarding micro processing technology related to manufacturing of a cell potential measuring device) can be considered as design matters by those having skills in the art based on the conventional art in the field of cell biology, physiology, medical science, pharmacy, biochemistry, genetic engineering, protein engineering, material science, semiconductor engineering, ultra-precision machining engineering, micro electro mechanical system (MEMS) engineering. The present technology can be carried out based on the description herein and the known technology in the above field.

### (Cell Potential Measuring Device)

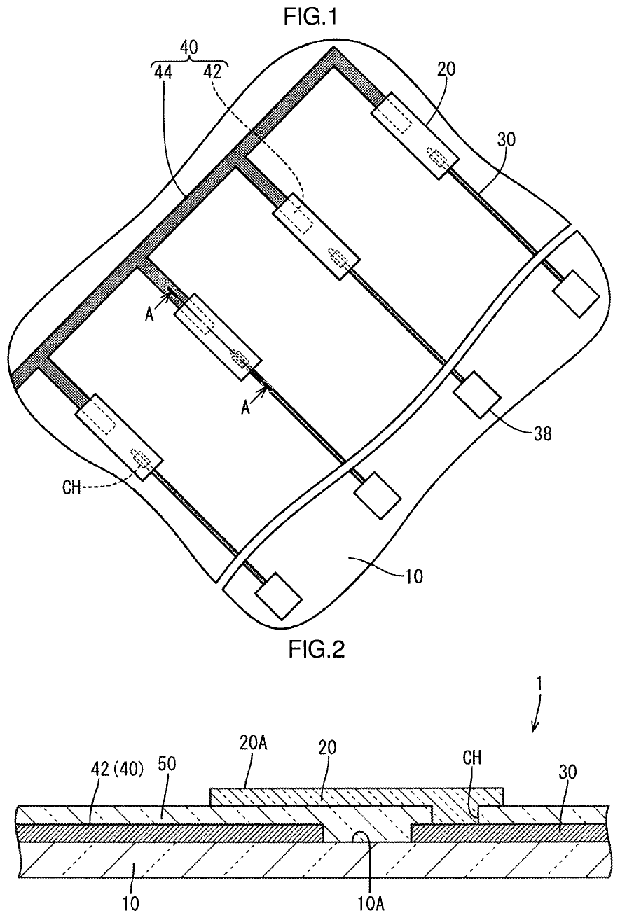

A cell potential measuring device described herein will be described with reference to FIGS. 1 to 5. The cell potential measuring device 1 is for recording electrical signals (action potentials), which are created by electrical activities of cells such as neuron, non-invasively outside the cells. As illustrated in FIGS. 1 and 2, the cell potential measuring device 1 includes a base plate 10, a measurement electrode 20 (a first electrode), a measurement line 30 (a first line), a test line 40 (a second line), and an insulation layer 50.

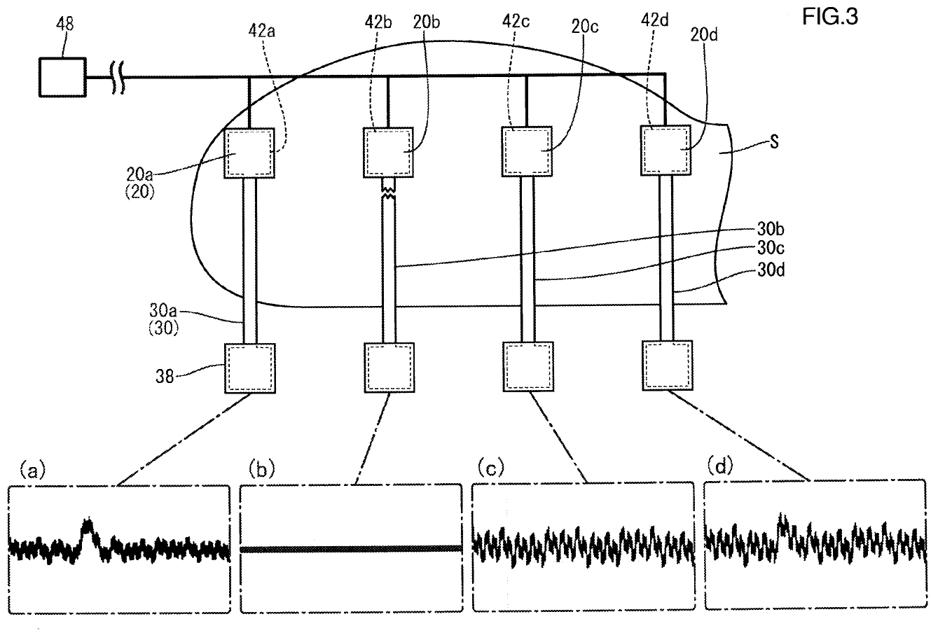

The base plate 10 supports the measurement electrodes 20, the measurement lines 30, the test line 40, and the insulation layer 50. The base plate 10 supports cells S that are an object to be measured (refer to FIG. 3) and is configured as a stage for cell seeding and cell culture of the cells S. The base plate 10 has a flat plate shape. The base plate 10 is made of insulating material having an electrically insulating property. Examples of the insulating material have volume resistivity at room temperature (for instance, 25° C.) being 10⁷ Ωcm or greater (for instance, 10¹⁰ Ωcm or greater, 10¹² Ωcm or greater, or 10¹⁵ Ωcm or greater). For example, the insulating material may be organic material or inorganic material having the above volume resistivity. The material of the base plate 10 is not limited to the above ones. The base plate 10 is preferably made of transparent material such that the cells S can be observed from below through the base plate 10. More preferably, the base plate 10 may be colorless and transparent.

Examples of the base plate 10 may be preferably various kinds of glass and synthetic resin. Preferable examples of glass may be soda-lime glass, borosilicate glass, and quats glass. The material of the base plate 10 is not necessarily limited to the above ones. The alkali-free glass that contains 0.1 mass % or less of alkaline component in terms of oxides and in which elution of alkali ions is highly suppressed may be used as the glass. Examples of synthetic resin include polydimethylsiloxane (PDMS), polystyrene, polypropylene, polyethylene terephthalate (PET), poly(methyl methacrylate), nylon, and polyurethane that have relatively high volume resistivity (for instance, 10¹⁰ Ωcm or more, 10¹² Ωcm or more, 10¹⁵ Ωcm or more) and biocompatibility. A thickness of the base plate 10 is not particularly limited but may preferably be about 0.2 mm to 1 mm (for instance, 0.5 mm, 0.7 mm).

The measurement electrodes 20 are configured to detect (receive) action potentials that are generated by the cells S. The measurement electrodes 20 are made of material having an electrically conductive property. Material of the measurement electrodes 20 will be described in detail later. The measurement electrode 20 has a layered structure and is disposed above the base plate 10. The plan view shape of the measurement electrode 20 is not limited and may be a linear shape, a rectangular shape, a square shape, a circular shape, and an irregular shape. The measurement electrode 20 of this embodiment has a rectangular plan view shape. At least a portion of an upper surface 20A of the measurement electrode 20 or preferably an entire area of the upper surface 20A may be configured as an exposed surface of the cell potential measuring device 1. One measurement electrode 20 or multiple measurement electrodes 20 may be disposed on one base plate 10. With multiple measurement electrodes 20 being disposed on one base plate 10, the action potential that is generated by the cell S can be detected with specifying a particular portion of the cell S where the action potential is generated. With multiple measurement electrodes 20 being disposed on one base plate 10, the measurement electrodes 20 may be preferably arranged regularly. The measurement electrode 20 is connected to the measurement line 30.

A shape and a size of the measurement electrode 20 is not particularly limited. In view of measuring the action potential of cells such as neuron, the measurement electrode 20 may have a rectangular shape having each side length of about 1 μm to 1000 μm (for instance, about tens of μm or more and 100 μm or less).

The measurement line 30 is for propagation of the action potential of the cell S that is received by the measurement electrode 20 to a connection portion that is connected to a potential measuring device, which is not illustrated. The measurement line 30 is electrically connected to the measurement electrode 20. One measurement line 30 is connected to one measurement electrode 20. With multiple measurement electrodes 20 being disposed on the base plate 10, one measurement line 30 is connected to at least one measurement electrode 20. The measurement lines 30 are preferably connected to the respective measurement electrodes 20. The measurement line 30 is made of material having an electrically conductive property. The material of the measurement line 30 will be described later in detail. The measurement line 30 is formed in a linear shape with an appropriate pattern. The measurement line 30 typically extends from a position on the base plate 10 where the measurement electrode 20 is to an edge portion of the base plate 10. An end portion of the measurement line 30 that is an opposite end from the end portion connected to the measurement electrode 20 may be connected to a connection terminal 38 that is wider than the measurement line 30 to ensure the connection to the potential measuring device. The upper surface of the measurement line 30 is typically covered by the insulation layer 50 such that the action potential received by the measurement electrode 20 is not applied to other portions of the cell S. The measurement line 30 is not necessarily limited to the above-described one but may be typically disposed directly on the base plate 10 (namely, without having any other layers therebetween).

The test line 40 is for testing and obtaining an electrical connection between the measurement electrode 20 and the measurement line 30. The test line 40 is made of material having an electrically conductive property such that the test signal can be sent to the measurement electrode 20. The material of the test line 40 will be described later in detail. The test line 40 includes a test electrode section 42 and a line section 44 that is electrically connected to the test electrode section 42.

The test electrode section 42 is disposed on the base plate 10 to be opposite the measurement electrode 20 with the test electrode section 42 being electrically insulated from the measurement electrode 20. The test electrode section 42 is typically disposed to be opposite the measurement electrode 20 via the insulation layer 50, which will be described later. With such a configuration, the test electrode section 42 and the measurement electrode 20 are configured as a capacitor and the test signal can be sent from the test line 40 to the measurement electrode 20. The shape of the test electrode section 42 is not particularly specified as long as the test electrode section 42 is opposite at least a portion of the measurement electrode 20 and at least a portion of the test electrode section 42 is opposite the measurement electrode 20. In this embodiment, the test electrode section 42 has a linear shape that is narrower than the measurement electrode 20. In view of forming a good capacitor with the measurement electrode 20, the test electrode section 42 may have each side length of about 1 μm to 500 μm and a length of about 1 μm to 1000 μm.

The line section 44 is for sending test signals output by a testing device, which is not illustrated, to the test electrode section 42. Typically, one line section 44 is connected to one test electrode section 42. The line section 44 is formed in a linear shape with an appropriate pattern. The line section 44 typically extends from a position of the base plate 10 where the test electrode section 42 is to an edge portion of the base plate 10. An end portion of the line section 44 that is an opposite end from the end portion connected to the test electrode section 42 may be connected to a connection terminal 48 that is wider than the line section 44 to ensure the connection to the testing device.

Typically, one test line 40 is connected to one measurement electrode 20. With the multiple measurement electrodes 20 being disposed on the base plate 10, the test line 40 is connected to at least one measurement electrode 20. The test line 40 is connected to one measurement electrode 20. The upper surface of the test line 40 is typically covered by the insulation layer 50 such that the test signal to be sent to the measurement electrode 20 is not applied to the cell S. The test line 40 of this embodiment is not necessarily limited to the above-described one but the test line 40 including the test electrode sections 42 and the line section 44 may be typically disposed directly on the base plate 10 (namely, without having any other layers therebetween).

The insulation layer 50 is at least for insulating the measurement electrode 20 from the test line 40. The insulation layer 50 is preferably for insulating upper surfaces and side surfaces of the measurement line 30 and the test line 40 from the outside. Therefore, the insulation layer 50 is substantially disposed in an area that corresponds to the measurement lines 30, the test line 40, and surrounding portions of the lines 30, 40 in a plan view. The insulation layer 50 may not be disposed in a portion of the upper surface 10A of the base plate 10 that is away from the measurement lines 30 and the test line 40 in a plan view. The portion of the upper surface 10A of the base plate 10 that is away from the measurement lines 30 and the test line 40 may be exposed to the outside. The measurement electrode 20 may be directly disposed on the portion of the upper surface 10A of the base plate 10 that is away from the measurement lines 30 and the test line 40. In each of the plan view drawings of the cell potential measuring device 1 of the present technology, outlines of the insulation layer 50 is not described for easy understanding of relative relation of the measurement electrode 20, the measurement line 30, and the test line 40. This is applied to the embodiments described below.

The insulation layer 50 is made of insulating material. Details of the material of the insulation layer 50 will be described later. The insulation layer 50 of this embodiment is disposed in the area where the measurement lines 30 and the test line 40 are disposed in a plan view and the surrounding area of the lines 30, 40 to cover the upper surfaces and the side surfaces of the measurement lines 30 and the test line 40. The insulation layer 50 of this embodiment is disposed under the measurement electrodes 20 in areas where the measurement lines 30 and the test line 40 are not disposed so as to support the measurement electrodes 20. A thickness of the insulation layer 50 that is between the measurement electrodes 20 and the test electrode sections 42 (the test line 40), which is a distance between the measurement electrodes 20 and the test electrode sections 42 (the test line 40), can be designed appropriately according to the relation of permittivity of the material of the insulation layer 50, an opposing area of the measurement electrode 20 and the test electrode section 42, and a capacitance required for a capacitor that is created by the measurement electrode 20 and the test electrode section 42. The distance between the measurement electrode 20 and the test electrode section 42 is preferably 10 nm or more (preferably 30 nm or more) and 100 μm or less (preferably 20 μm or less), for instance.

In this s embodiment, as described before, the measurement lines 30 and the test line 40 are disposed on the base plate 10 without having any other layers therebetween. The measurement electrodes 20 are disposed above the test line 40 via the insulation layer 50. The insulation layer 50 has contact holes CH. With the contact hole CH being filled with the material of the measurement electrode 20, the measurement electrode 20 and the measurement line 30 are electrically connected. Namely, the cell potential measuring device 1 is configured as a layered structure body that includes an electrically conductive layer and the insulation layer 50. The electrically conductive layer includes the measurement electrodes 20, the measurement lines 30, and the test line 40.

The electrically conductive layer (the measurement electrodes 20, the measurement lines 30, and the test line 40) can be made of electrically conductive material having an electrically conductive property. Examples of such electrically conductive material include metal material, electrically conductive resin material, and electrically conductive inorganic material. The metal material is preferable since the metal material is good in thermal stability and electrical conductivity. Examples of the metal material may be one kind selected from the metals such as gold (Au), silver (Ag), copper (Cu), titanium (Ti), aluminum (Al), nickel (Ni), Cr (chrome), molybdenum (Mo), niobium (Nb), Ta (tantalum), and tungsten (W) or an alloy including the selected metal, or an alloy including two kinds or more selected from the metals. Since the metal including such an element has high electrical conductivity, resistivity can be decreased even with fine electrodes and lines being made of such metal. Preferable examples of the metal material include Au, Ag, Cu, W, Ti, Al, TaN (tantalum nitride), and MOW (molybdenum-tungsten alloy). The electrically conductive layer (for example, the measurement line 30 and the test line 40) that is disposed closer to the base plate 10 and a portion of the electrically conductive layer that is contacted with the base plate 10 may preferably be made of metal having a relatively high melting point such as Ta, W, Mo, Ni, Ti. A portion relatively farther away from the base plate 10 may be preferably made of metal having relatively low resistance such as Au, Al, Cr to avoid signal degradation. To decrease the electrical resistance, for instance, the electrically conductive layer may have a single-layer structure made of MoW alloy having a low resistance or a multi-layer structure including W/TaN, Ti/Al/Ti, Cu/Ti disposed from an upper layer side such that good close contact with a base member (such as a base plate) and low electrical resistance can be achieved. Preferable examples of metal material for the electrically conductive layer included in a portion that can be contacted with the cells S are Au and Ti having low cytotoxicity. The measurement lines 30 and the test line 40 of this embodiment are made of such metal material.

Preferable examples of the electrically conductive resin material include electrically conductive polyacetylene, electrically conductive polythiophene, electrically conductive polyaniline, and electrically conductive PEDOT. Preferable example of electrically conductive inorganic material may be semiconductive oxides (may be metal oxide) having a band gap of 3 eV or more. Examples of such semiconductive oxides include tin oxide (SnO₂: including tin oxide to which Sb (antimony), Ta, F (fluorine) is added), zinc oxide (ZnO: including zinc oxide to which Al, Ga (gallium) is added), indium tin oxide (ITO), indium zinc oxide (IZO), and indium gallium zinc oxide. It is confirmed such semiconductive oxides are transparent and bioinert. As will be described in a subsequent embodiment, the measurement electrode 20 may be made to have a greater area than the measurement line 30 and the test line 40. In such a configuration, the measurement electrode 20 is preferably made of transparent material with respect to visible light similar to the base plate 10. With such a configuration, the cells S to be cultured can be observed from a lower surface of the base plate 10 without obstruction by the measurement electrode 20. For instance, the measurement electrodes 20 of this embodiment are made of ITO. With such material being used, a stable electrically conductive layer having low cytotoxicity can be obtained.

The insulation layer 50 may be made of material having electrically insulation property similar to the base plate 10. Material that exerts stable electrical insulation property in the cell culture environment can be used without any limitation. The insulation layer 50 may be preferably made of silicon nitride (Si₃N₄, for instance), silicon dioxide (SiO₂, for instance), and silicon oxynitride (Si₂N₂O, for instance) with considering the feature of the insulation layer 50 that is contacted with the measurement electrode 20, the measurement line 30, and the test line 40 and exerts the mutual insulation. However, the material of the insulation layer 50 is not limited to the above ones. The insulation layer 50 may have a single-layer structure made of one of the above materials or a multi-layer structure made of two or more kinds of the above materials. Regarding the above materials, typical compositions are illustrated in the parenthesis; however, the composition of each material is not limited thereto.

### (Producing Method)

A method of producing the above cell potential measuring device 1 is not particularly limited and may be preferably produced with following steps, for instance. At first, the base plate 10, which is a non-alkaline glass plate, is prepared and the measurement lines 30 and the test line 40 are formed on one surface (the upper surface 10A) of the base plate 10 in a predefined pattern. In the configuration including the connection terminals 38, 48, the connection terminals 38, 48 are formed at the same time as the measurement lines 30 and the test line 40 are formed. The measurement lines 30 and the test line 40 are preferably formed as described below, for instance. After the electrically conductive film made of the above-described metal material is formed on the entire area of the upper surface 10A of the base plate 10 with the sputtering method or the vapor deposition method, the electrically conductive film is processed with lithography (photolithography, laser lithography, for instance) into a predefined pattern and the measurement lines 30 and the test line 40 are formed. With the photolithography, a film to be patterned (the electrically conductive film in this embodiment) is subjected to resist coating, light exposure, and rinse to form a photoresist pattern. The electrically conductive film is subjected to etching with using the photoresist pattern as the mask and the portions of the electrically conductive film that are not covered by the mask are removed. Thus, the electrically conductive film having a desired pattern can be obtained. To reduce the electrical resistance, for instance, the measurement lines 30 and the test line 40 may have a single-layer structure made of MoW alloy having a low resistance or a multi-layer structure made of W/TaN or Ti/Al/Ti. The thickness of the measurement lines 30 and the thickness of the test line 40 are not particularly limited and may be 10 nm or more (preferably 30 nm or more) and 1 μm or less (preferably 500 nm or less) for instance.

Next, upper surfaces of the base plate 10, the measurement lines 30 and the test line 40, which are formed on the base plate 10 are covered by the insulation layer 50. In a plan view, the insulation layer 50 that is formed in areas except for the measurement electrodes 20, the measurement lines 30, the test line 40, and the surrounding portions thereof is removed with etching. The insulation layer 50 that is formed on the connection terminals 38, 48 is also removed with etching. A portion of the insulation layer 50 that covers each measurement line 30 is partially removed to form the contact hole CH and expose the measurement line 30. Accordingly, the surfaces of the measurement lines 30 and the test line 40 are selectively covered by the insulation layer 50. The thickness of the insulation layer 50 is not particularly limited and may be 10 nm or more (preferably 30 nm or more) and 100 μm or less (preferably 20 μm or less) for instance.

Then, the measurement electrodes 20 are formed on the insulation layer 50 in a predefined shape with the contact holes CH being filled with the measurement electrodes 20. The measurement electrodes 20 are made of transparent electrically conductive material such as ITO. The layer of transparent electrically conductive material may be formed with sputtering, for instance. In the configuration including the connection terminals 38, 48, a layer of transparent electrically conductive material may be preferably formed on the connection terminals 38, 48 with sputtering. The thickness of the transparent electrically conductive material (namely, the measurement electrode 20) may be about 10 nm (preferably 30 nm or more) and about 300 nm or less (preferably 100 nm or less) for instance. With such a configuration, the layer of metal material is not disposed on an outermost side and not exposed to outside and the cell potential measuring device 1 having good chemical stability can be produced.

### Operations and Effects

In the above embodiment, the cell potential measuring device 1 includes the base plate 10 having an insulating property, the measurement lines 30 that are disposed on the base plate 10, the insulation layer 50 that is disposed on the base plate 10 and covers at least the surfaces of the measurement lines 30, the measurement electrodes 20 that are disposed on the insulation layer 50 and electrically connected to the measurement lines 30, and the test line 40 that is disposed on the base plate 10 and at least a portion of which is disposed under the measurement electrode 20 via the insulation layer 50.

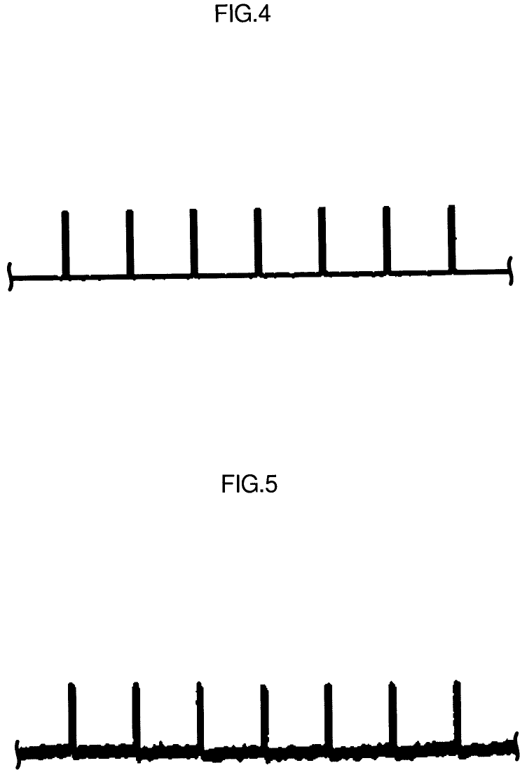

With using such a cell potential measuring device 1, the electrical connection of the measurement electrodes 20 and the measurement lines 30 can be tested. Specifically, as illustrated in FIG. 3, in measuring action potentials of cells, the test signal having a pulse waveform illustrated in FIG. 4 is previously input to the test electrode sections 42 from a testing device, which is not illustrated, via the connection terminal 48 and the line sections 44. Since the measurement electrode 20 and the test electrode section 42 (the test line 40) are configured as a capacitor, the electrical insulation is maintained between the measurement electrode 20 and the test line 40 but the test signals can be transferred therebetween. For instance, when the test electrode section 42 is electrically charged by the test signal, the measurement electrode 20 is electrically charged with a polarity opposite from that of the electric charge of the test electrode section 42. When the test electrode section 42 has no electric charge, the measurement electrode 20 also has no electric charge accordingly. Therefore, the test signal is transferred to the measurement electrode 20. With the electrical connection between the measurement electrodes 20 and the measurement lines 30 via the contact hole portions being good and no disconnection occurring in the measurement electrodes 20 and the measurement lines 30, the test signals from the measurement electrodes 20 are detected by a potential measuring device, which is not illustrated, via the measurement lines 30 and the connection terminals 38. As a result, it is confirmed that no electrical error occurs in the measurement electrodes 20 and the measurement lines 30 and a good electrically conductive state is maintained. For instance, s illustrated in FIG. 3, disconnection occurs in the as measurement line 30b. In such a configuration, even if the test signal is input to the test line 40, no test signal is detected from the measurement line 30b. Thus, it can be confirmed that the measurement electrode 20 and the measurement line 30 are not electrically conductive when the potential measuring device detects no response test signal.

Furthermore, by comparing the input waveform of the test signal input to the test line 40 (refer to FIG. 4) and the output waveform of the test signal output from the measurement line 30 (refer to FIG. 5), electrical characteristics of the measurement electrode 20 and the measurement line 30 such as resistance, propagation delay, signal attenuation can be obtained. For instance, as the electrically conductive state of the measurement electrodes 20a to 20d and the measurement lines 30a to 30d is better, the shape and size of the output waveform become closer to those of the input waveform. On the other hand, as the electrically conductive state of the measurement electrodes 20a to 20d and the measurement lines 30a to 30d is worse, the shape of the output waveform largely changes from the input waveform and the attenuation may become larger. Thus, by comparing the output waveform from each of the measurement lines 30a to 30d and the input waveform of the test signal, the electrically conductive state of the measurement electrodes 20a to 20d and the measurement lines 30a to 30d can be evaluated.

The results of the action potentials of the cells S measured by the cell potential measuring device 1 are typically illustrated with the graphs (a) to (d) in a lower section of FIG. 3. To simplify the description, it is supposed that the same action potential is input from the cells S to each of the measurement electrodes 20a to 20d. The test signal is not detected in the graph (b) in FIG. 3 that represents the measurement result of the measurement electrode 20b that is connected to the disconnected measurement line 30b. With the cell potential measuring device 1, it can be known by the previously performed electrode test that the electrically conductive state of the measurement electrode 20b and the measurement line 30b is not good. Therefore, the reason no test signal is detected in the graph (b) is not that the cells S locally do not create an action potential at the measurement electrode 20b but that an error in electrical conductivity such as disconnection occurs in the measurement electrode 20b and the measurement line 30b. It can be known that a relatively large test signal is detected in the graph (a) because the cells S locally create an action potential at the measurement electrode 20a.

The specific activity of the cells S, which is not specifically illustrated, can be evaluated by performing chronological analysis of the signals detected in the graphs (a) (c), (d). Specifically, for instance, with information being sent from one neuron to another neuron in living tissue, steep electrical change (impulse) of 1 millisecond or less is caused. It has been known that the action potential is not affected by how strong the stimulus caused by the information transfer is and the number of impulses that are generated per a unit time is increased as the stimulus caused by the information transfer becomes stronger. With using the cell potential measuring device 1 according to the present technology, such information transfer between the neurons can be recognized by the chronological and two-dimensional conversion into numerical values. More precise measurement of the cell activity can be performed by using as feedback the evaluation results of the electrical characteristics of the measurement electrodes 20 and the measurement lines 30 (such as signal attenuation and propagation delay) for the analysis of the action potentials of cells.

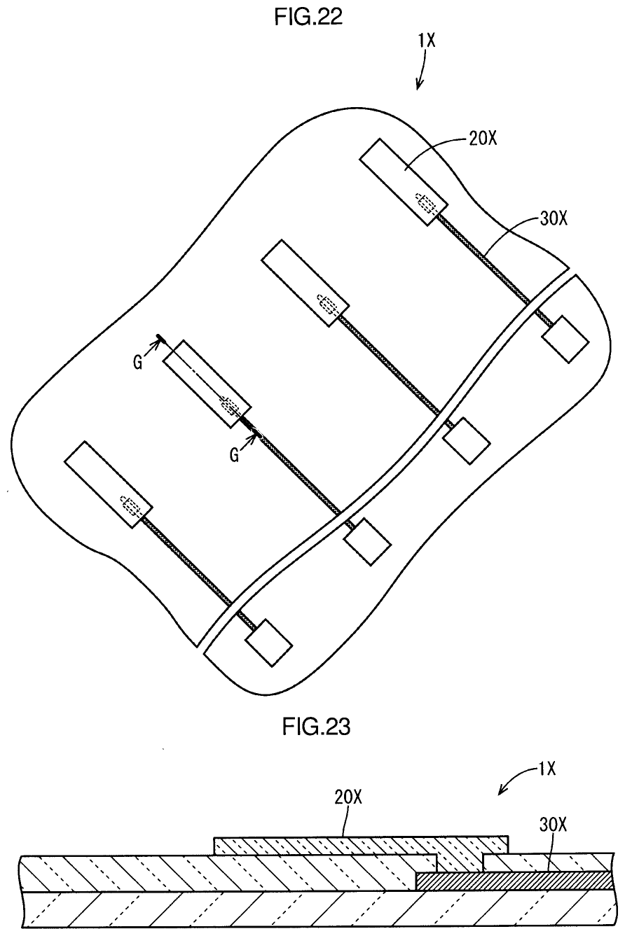

In a cell potential measuring device 1X (refer to FIGS. 22, 23) of a prior art including no test lines, when a test signal is input to a test line and a desired detection result is not obtained via a measurement line 30X, it cannot be determined whether the reason thereof is that the cell potential measuring device 1X has a problem or that the cell itself has a problem. The cell potential measuring device 1 of this embodiment can obtain the electrically conductive state, which includes confirmation of correct electrical connection from the measurement electrode 20 to the measurement line 30, by the signal that is detected via the measurement line 30 when the test signal is input to the test line 40. Accordingly, it can be tested whether the cell potential measurement can be properly performed. Furthermore, more precise analysis of the detection signals that are detected via the measurement line 30 can be performed. With such a configuration, the effects may be apparently exerted when the number of electrodes is increased to measure the actions of cells with higher sensitivity and more in detail.

In this embodiment, the test line 40 includes the test electrode section 42 that is disposed opposite the measurement electrode 20 and the line section 44 that extends from the test electrode section 42. The distance between the measurement electrode 20 and the test electrode section 42 is 10 nm or more and 100 μm or less. With such a configuration, the capacitor structure including the measurement electrode 20 and the test electrode section 42 (the test line 40) can be preferably configured for obtaining the electrically conductive state of the measurement electrode 20 and the measurement line 30.

In this embodiment, the insulation layer 50 covers the surface of the test line 40. With such a configuration, even if the cells S that are to be observed and disposed on the cell potential measuring device 1 are disposed above the test line 40, the test line 40 is insulated from the cells S. As a result, the action potentials generated by the cells S are less likely to propagate to the test line 40. In other words, the action potentials generated by the cells S are less likely to be received by the test line 40 and less likely to propagate to the measurement electrodes 20 and the measurement lines 30 as a noise. Furthermore, when the test signals are sent via the test line 40 to obtain the electrically conductive state of the measurement electrodes 20 and the measurement lines 30, the test signals are less likely to be applied to the cells S as the electrical stimulus. Accordingly, unintentional electrical stimulus is less likely to be applied to the cells S. Therefore, action potentials of the cells S can be measured more correctly. According to such a configuration, the cell potential measuring device 1 that includes multiple sets each of which includes the measurement electrode 20, the measurement line 30, and the test line 40 can specify a portion of the cells that generates the detected action potential quite precisely.

In this embodiment, the test line 40 is entirely disposed directly on the base plate 10. According to such a configuration, in producing the cell potential measuring device 1 with lithography, for example, the test line 40 can be made of the same material and formed in the same process as that of the measurement line 30 on the base plate 10. The lithography is preferable because the test line 40 can be formed with using the metal material having good electrical conductivity by directly providing the test line 40 on the base plate 10.

In this embodiment, the measurement electrodes 20 are made of ITO (one example of transparent electrically conductive material). With such a configuration, the measurement electrodes 20 can be made transparent. For instance, with an area of the measurement electrode 20 being increased and a portion of the measurement electrode 20 being directly disposed on the glass base plate 10, transparency of the portion of the glass base plate 10 where the measurement electrode 20 is directly disposed can be maintained. As a result, in measuring the action potentials of the cells S, the cells S can be observed from a lower surface of the glass base plate 10 through the transparent measurement electrode 20. Furthermore, ITO is preferably used because ITO is electrically conductive inorganic material and has low cytotoxicity and is also chemically stable with respect to the cell culture environment.

In this embodiment, the measurement electrodes 20 include measurement electrodes 20a to 20d (an example of a first measurement electrode (a third electrode) and an example of a second measurement electrode (a fourth electrode)). The test line 40 includes the test electrode section 42 (a second electrode section) that is opposite the measurement electrode 20 and the line section 44 that extends from the test electrode section 42. The test electrode section 42 includes test electrode sections 42a to 42d (an example of a first test electrode section (a third electrode section) and an example of a second test electrode section (a fourth electrode section)) that are opposite the measurement electrodes 20a to 20d, respectively. The test electrode sections 42a to 42d are connected to one line section 44. According to such a configuration, in one cell potential measuring device 1, the action potentials of the cells can be measured with using two or more measurement electrodes 20 and electrically conductive state of the measurement electrodes 20 and the corresponding measurement lines 30 can be tested. With the test electrode sections 42a to 42d being connected to one line section 44, the test signals can be easily input. Therefore, the action potentials of multiple cells can be measured easily, for instance. Furthermore, action potentials of different portions of one cell can be measured easily.

### Second Embodiment

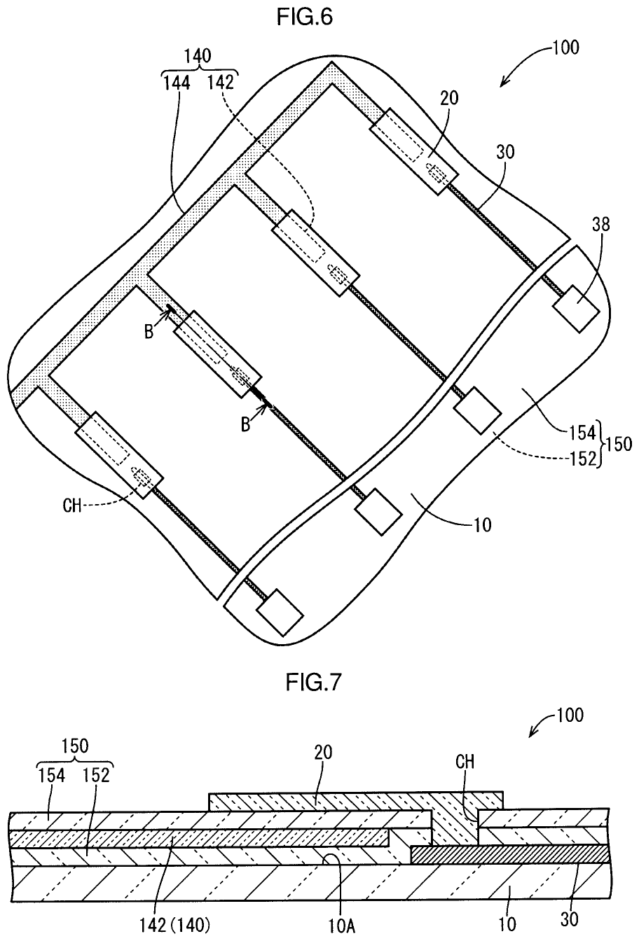

A second embodiment will be described with reference to FIGS. 6 and 7. In the first embodiment, the test electrode section 42 and the line section 44 of the test line 40 are directly disposed on the base plate 10. The insulation layer 50 is configured as one layer (one layer may have a multi-layered structure). In a cell potential measuring device 100 of the second embodiment, an insulation layer 150 includes a first insulation layer 152 and a second insulation layer 154. The first insulation layer 152 is disposed on the upper surface 10A of the base plate 10. The second insulation layer 154 is disposed on an upper side than the first insulation layer 152. A test electrode section 142 and a line section 144 of a test line 140 are disposed on the first insulation layer 152. Other configurations, operations, and effects are similar to those of the first embodiment and will not be described.

The first insulation layer 152 is disposed in an area that corresponds to the measurement electrodes 20, the measurement lines 30, the test line 140, and surrounding portions thereof in a plan view. In the portions including the measurement lines 30, the first insulation layer 152 covers the measurement lines 30 from above. In the portions including the test line 140, the first insulation layer 152 is disposed between the base plate 10 and the test line 140. The second insulation layer 154 is disposed in an area that corresponds to the measurement electrodes 20, the measurement lines 30, the test line 140, and surrounding portions thereof in a plan view. In the portions including the test line 140, the second insulation layer 154 covers the test line 140 from above. In the portions including no test line 140, the second insulation layer 154 covers the first insulation layer 152 from above. The second insulation layer 154 is disposed between the test electrode section 142 and the measurement electrode 20. Each of the first insulation layer 152 and the second insulation layer 154 can be made of insulating material described in the first embodiment. Each of the first insulation layer 152 and the second insulation layer 154 may have a multi-layer structure.

### (Producing Method)

The cell potential measuring device 100 may be preferably produced with the known lithography technology similar to the first embodiment. First, the measurement lines 30 are formed on one surface (the upper surface 10A) of the base plate 10 with a predefined pattern form. The measurement lines 30 of this embodiment include a multi-layer structure including aluminum (such as layers of Ti/Al/Ti in this order from an upper layer). Next, the second insulation layer 154 is disposed on upper surfaces of the first insulation layer 152 and the test line 140. Then, portions of the first insulation layer 152 and the second insulation layer 154 that cover the measurement line 30 are removed to form the contact hole CH and expose the measurement line 30. The measurement electrodes 20 are formed on the upper surface of the second insulation layer 154 in a predefined shape such that the contact holes CH are filled with the measurement electrodes 20. Thus, the measurement electrodes 20 and the measurement lines 30 are electrically conductive and the cell potential measuring device 100 is obtained.

With the above configuration, the test electrode sections 142 can be disposed below the measurement electrodes 20 so as not to be contacted with the base plate 10. Accordingly, wide variety of materials of the test electrode sections 142 (the test line 140) can be used. For instance, in producing the cell potential measuring device 100 with lithography, heat is less likely to be transferred from the base plate 10 to the test line 140. Therefore, the test line 140 may be preferably made of material that is relatively low in heat stability and base plate followability but has low resistance.

In the above embodiment, the measurement lines 30 and the test line 140 have a multi-layer structure of Ti/Al/Ti from the upper layer and a multi-layer structure of Ti/Al/Ti from the upper layer, respectively. With such a configuration, a line width of the measurement line 30 and the test line 140 can be reduced and a thickness of the lines 30, 140 can be increased to reduce a resistance.

### Third Embodiment

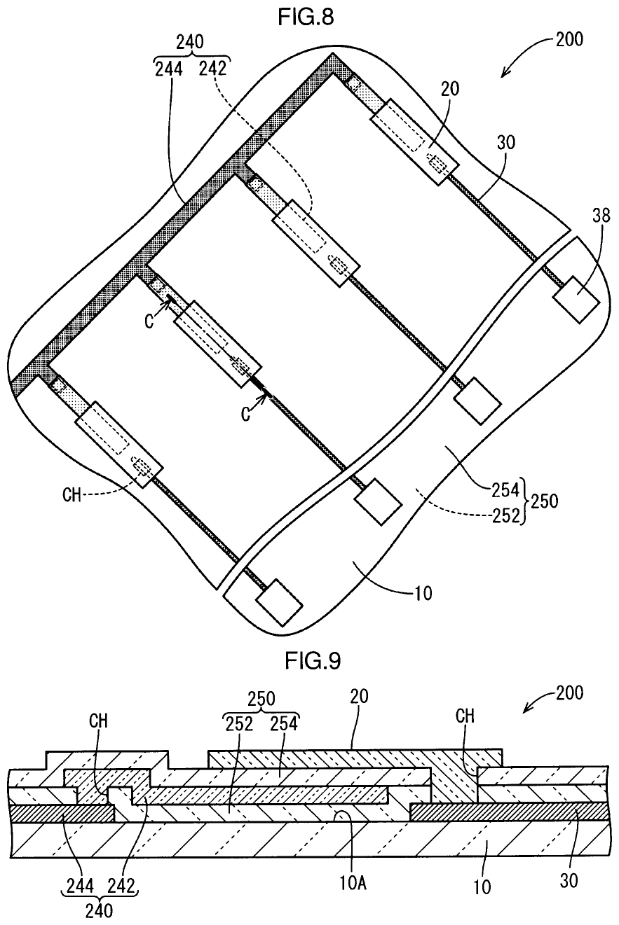

A third embodiment will be described with reference to FIGS. 8 and 9. In the first and second embodiments, the test electrode sections 42, 142 and the line sections 44, 144 of the test line 40, 140 are formed on the base plate 10 or the first insulation layer 152 in the same process (in the same layer level). The test electrode sections 42, 142 and the line sections 44, 144 are made of the same material. In a cell potential measuring device 200 of the third embodiment, configurations of an insulation layer 250 and a test line 240 differ from those of the above embodiments. Other configurations, operations, and effects are similar to those of the first and second embodiments and will not be described.

The test line 240 includes a test electrode section 242 that is opposite the measurement electrode 20 and a line section 244 that extends from the test electrode section 242. Similar to the measurement line 30, the line section 244 is disposed directly on the base plate 10. The test electrode section 242 is disposed on the insulation layer 250. The insulation layer 250 includes a first insulation layer 252 that is disposed between the base plate 10 and the test electrode section 242 and a second insulation layer 254 that is disposed between the test electrode section 242 and the measurement electrode 20. The first insulation layer 252 is disposed in an area that corresponds to the measurement electrodes 20, the measurement lines 30, the line sections 244, and surrounding portions thereof in a plan view. In the portions including the measurement lines 30 and the line sections 244, the first insulation layer 252 covers the measurement lines 30 and the line sections 244 from above. In the portions including no measurement lines 30 and no line sections 244, the first insulation layer 252 is contacted with the base plate 10. The test electrode sections 242 are disposed on the first insulation layer 252 and below the measurement electrodes 20. The second insulation layer 254 is disposed in an area that overlaps the first insulation layer 252 in a plan view. In the portions where the test electrode sections 242 are on the first insulation layer 252, the second insulation layer 254 covers the test electrode sections 242 from above. In the portions where no test electrode sections 242 are disposed, the second insulation layer 254 is contacted with the first insulation layer 252. The test electrode section 242 and the line section 244 are connected via the contact hole CH.

### Producing Method

The cell potential measuring device 200 may be preferably produced with the known lithography technology similar to the first embodiment. First, the measurement lines 30 and the line sections 244 are formed on one surface (the upper surface 10A) of the base plate 10 with a predefined pattern form. The measurement lines 30 and the line sections 244 of this embodiment are made of metal material. Next, the first insulation layer 252 is disposed on upper surfaces of the base plate 10, the measurement lines 30, and the line sections 244. Then, portions of the first insulation layer 252 that covers the line sections 244 are partially removed to form the contact holes CH and expose the line sections 244. The test electrode sections 242 are formed on the upper surface of the first insulation layer 252 in a predefined pattern form such that the contact holes CH are filled with the test electrode sections 242. The test electrode sections 242 of this embodiment are made of metal material. Accordingly, the test electrode sections 242 and the line sections 244 are connected via the contact holes CH. Next, the second insulation layer 254 is disposed on upper surfaces of the first insulation layer 252 and the test electrode sections 242. Then, portions of the first insulation layer 252 and the second insulation layer 254 that cover the measurement lines 30 are partially removed to form the contact holes CH and expose the measurement lines 30. The measurement electrodes 20 are formed on the upper surface of the second insulation layer 254 in a predefined pattern form such that the contact holes CH are filled with the measurement electrodes 20. The measurement electrodes 20 of this embodiment are made of transparent electrically conductive material (such as ITO). Thus, the measurement electrodes 20 and the measurement lines 30 are electrically conductive and the cell potential measuring device 200 is obtained.

With the above configuration, the first insulation layer 252 and the second insulation layer 254 are disposed on the line sections 244 of the test line 240 and the measurement lines 30. Therefore, the line sections 244 and the measurement lines 30 are surely insulated from the cells S such that the action potentials do not propagate from the cells S that are to be observed. The line sections 244 can be made of material different from that of the test electrode section 242, and the line sections 244 and the measurement lines 30 are disposed on the base plate 10. Therefore, the line sections 244 of the test line 240 can be made of highly electrically conductive metal material. The test electrode sections 242 are disposed below the measurement electrodes 20 so as not to be contacted with the base plate 10. The test electrode sections 242 may be made of transparent electrically conductive material (such as ITO). With such a configuration, the cells S can be observed from a lower surface of the base plate 10 without obstruction by the measurement electrodes 20 and the test electrode sections 242. Therefore, testing of the electrodes and measuring of the action potentials of cells can be performed more precisely.

### Fourth Embodiment

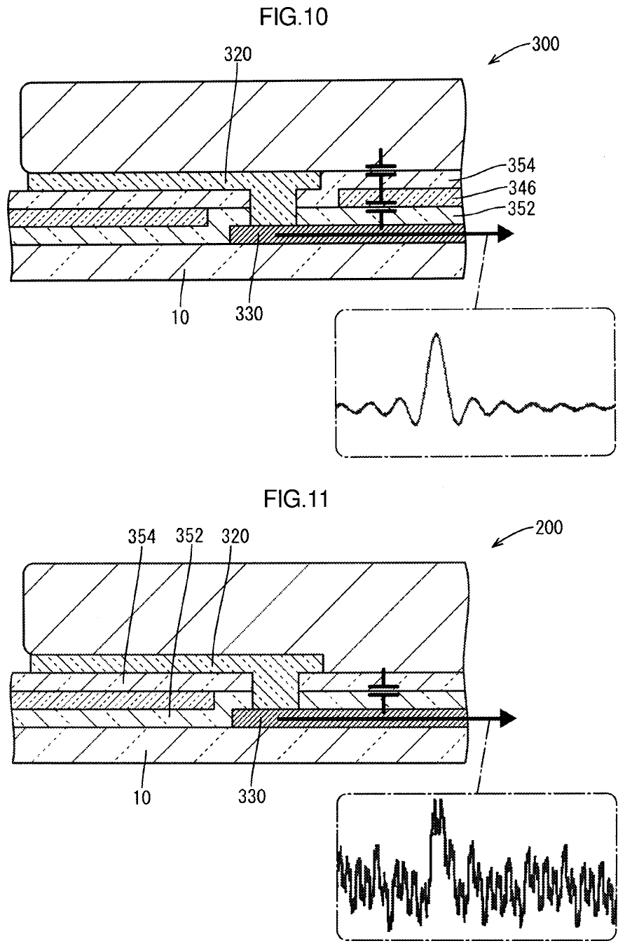

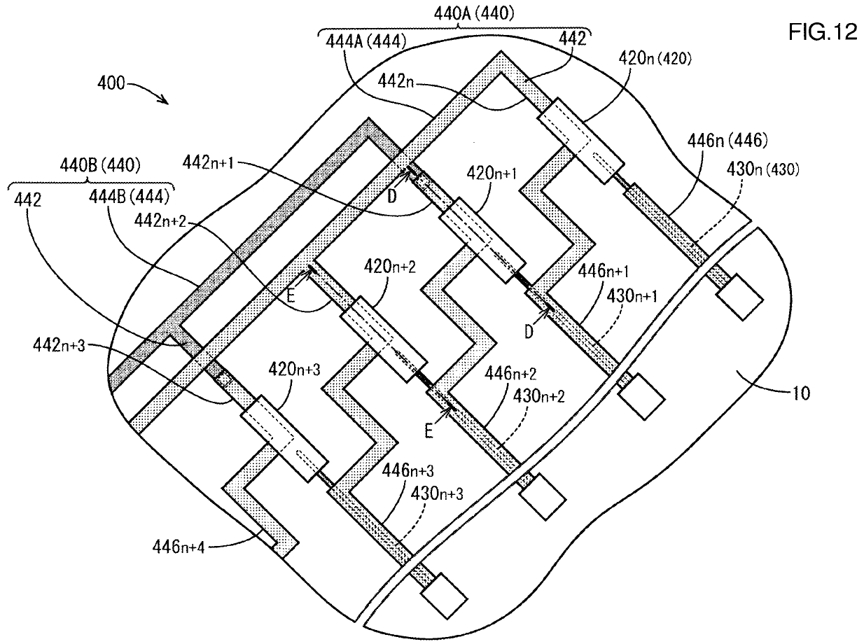

A fourth embodiment will be described with reference to FIGS. 10 and 11. In the second and third embodiments, the second insulation layers 154, 254 are disposed directly on the first insulation layers 152, 252, respectively, above the measurement lines 30. A cell potential measuring device 300 of the fourth embodiment differs from the above embodiments in that a shield layer 346 is disposed between a first insulation layer 352 and a second insulation layer 354 above a measurement line 330. Other configurations, operations and effects may be same as those of the first to third embodiments and will not be described.

The shield layer 346 is made of electrically conductive material having electrical conductivity. The shield layer 346 is disposed between the first insulation layer 352 and the second insulation layer 354 above the measurement line 330. The shield layer 346 is for shielding the measurement lines 330 from noise. The shield layer 346 is insulated from measurement electrodes 320. The shield layer 346 has a wide plan view shape so as to cover the measurement lines 330 in a width direction. The shield layer 346 may be connected to a ground potential but is not necessarily limited thereto.

When the cell culture environment such as cell culture liquid and cell culture medium is on the cell potential measuring device 300, the action potentials generated by the cells may propagate to the measurement line 330 via the cell culture liquid or medium and may be detected as a noise. In the above configuration, the shield layer 346 is between the first insulation layer 352 and the second insulation layer 354. Therefore, the second insulation layer 354 and the shield layer 346, which form a multi-layer structure, is configured as a capacitor. The shield layer 346, the first insulation layer 352, and the measurement line 330, which form a multi-layer structure, is configured as another capacitor. With such a configuration, noise propagation from the cell culture environment to the measurement line 330 can be effectively suppressed.

### Fifth Embodiment

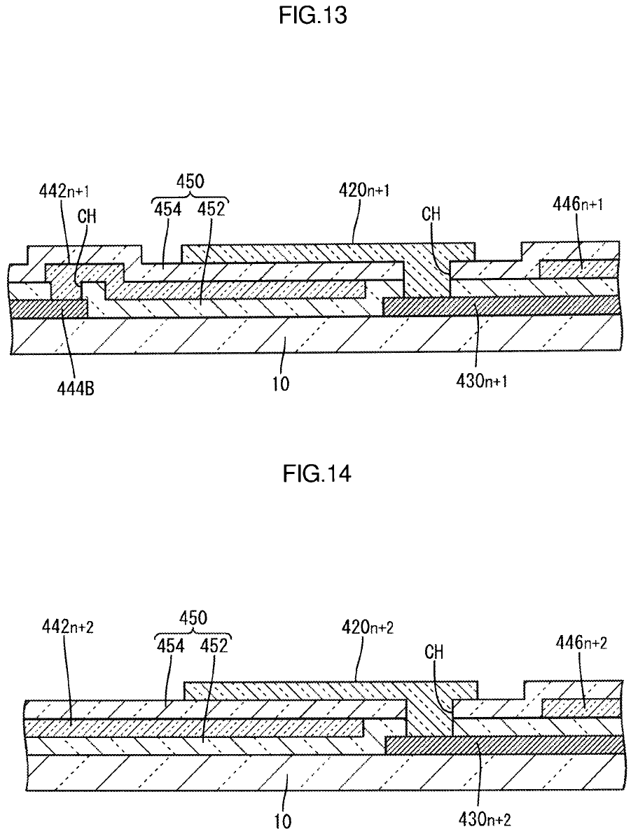

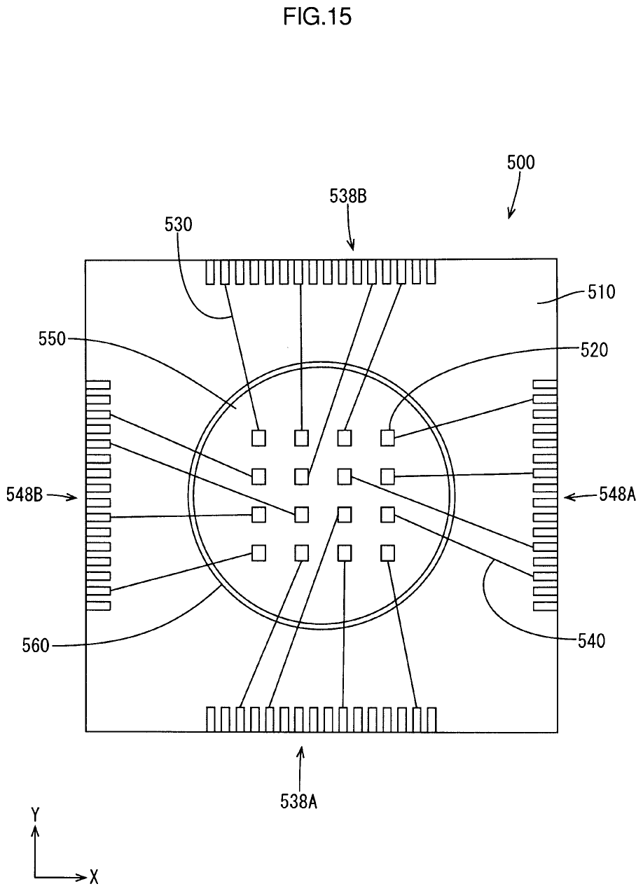

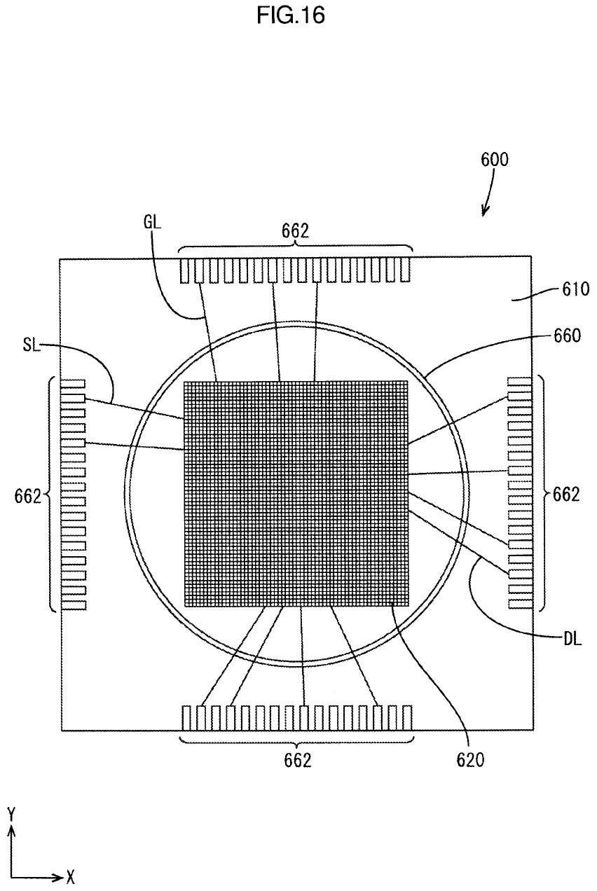

A fifth embodiment will be described with reference to FIGS. 12 to 14. In the fourth embodiment, the test line 340 has a single line structure that includes multiple test electrode sections 342 that are connected to one line section and the measurement lines 30 are shielded by the shield layer 346. A cell potential measuring device 400 according to the fifth embodiment differs from the fourth embodiment in that a test line 440 has a two-lines structure (a first test line 440A and a second test line 440B) and the cell potential measuring device 400 includes a shield layer 446 having a configuration different from that of the fourth embodiment. Other configurations, operations and effects may be same as those of the first to fourth embodiments and will not be described.

The shield layers 446 of this embodiment are connected to test electrode sections 442, respectively. In other words, the shield layers 446 are extended portions of the respective test electrode sections 442. However, with the shield layer 446 and the test electrode section 442 being simply connected, the test signal is also sent to measurement line 430 when electrical conductivity of measurement electrode 420 and the measurement line 430 is tested. Therefore, it cannot be determined whether the measurement electrode 420 and the measurement line 430 are effectively contacted. The test line 440 of the cell potential measuring device 400 has the following configuration.

The cell potential measuring device 400 includes measurement electrodes 420ₙ, 420ₙ₊₁, 420ₙ₊₂, 420ₙ₊₃ . . . (n is an integer of 1 or greater). To generally referring to the measurement electrodes 420ₙ/420ₙ₊₁, 420ₙ₊₂, 420ₙ₊₃, the measurement electrodes are simply referred to the measurement electrodes 420. The measurement electrodes 420 are arranged along one direction. The measurement electrodes 420 are defined into first measurement electrodes 420ₙ, 420ₙ₊₂ . . . and second measurement electrodes 420ₙ₊₁, 420ₙ₊₃ . . . that are arranged alternately. The measurement electrodes 420ₙ, 420ₙ₊₁, 420ₙ₊₂, 420ₙ₊₃ . . . are connected to measurement lines 430ₙ, 430ₙ₊₁, 430ₙ₊₂, 430ₙ₊₃ . . . , respectively.

The measurement lines 430 are defined into first measurement lines 430ₙ, 430ₙ₊₂ . . . (third lines) that are connected to the first measurement electrodes 420ₙ, 420ₙ₊₂ . . . , respectively, and second measurement lines 430ₙ₊₁, 430ₙ₊₃ . . . (fourth lines) that are connected to the second measurement electrodes 420ₙ₊₁, 420ₙ₊₃ . . . , respectively. The shield layers 446 are defined into first shield layers 446ₙ, 420ₙ₊₂ . . . that are for shielding the first measurement lines 430ₙ, 430ₙ₊₂ . . . from above and second shield layers 446ₙ₊₁, 446ₙ₊₃ . . . that are for shielding the second measurement lines 430ₙ₊₁, 430ₙ₊₃ . . . from above.

The test line 440 includes a test electrode section 442 that is opposite the measurement electrode 420 and a line section 444 that extends from the test electrode section 442.

The test electrode section 442 includes first test electrode sections 442, 442ₙ₊₂ . . . , which are opposite the first measurement electrodes 420ₙ, 420₊₂ . . . , respectively, and second test electrode sections 442ₙ₊₁, 442ₙ₊₃ . . . , which are opposite the second measurement electrodes 420ₙ₊₁, 420ₙ₊₃ . . . , respectively. The line section 444 includes a first line section 444A that is connected to the first test electrode sections 442ₙ, 442ₙ₊₂ . . . and a second line section 444B that is connected to the second test electrode sections 442ₙ₊₁, 442ₙ₊₃ . . . . The first test electrode sections 442ₙ, 442ₙ₊₂ . . . and the first line section 444A have a configuration similar to that of the test line 140 of the second embodiment. The second test electrode sections 442ₙ₊₁, 442ₙ₊₃ . . . and the second line section 444B have a configuration similar to that of the test line 240 of the third embodiment. Namely, the test line 440 includes the first test line 440A, which includes the first line section 444A, and the second test line 440B, which include the second line section 444B.

The first measurement electrodes 420ₙ, 420ₙ₊₂ . . . of the first test line 440A and the second measurement electrodes 420ₙ₊₁, 420ₙ₊₃ . . . of the second test line 440B are arranged alternately along one direction on the surface of the base plate 10. In other words, the first test electrode sections 442ₙ, 442ₙ₊₂ . . . and the second test electrodes sections 442ₙ₊₁, 442ₙ₊₃ . . . are arranged alternately along one direction in a plan view.

The first line section 444A of the first test line 440A and the measurement lines 430 are disposed directly on the base plate 10. An insulation layer 450 includes a first insulation layer 452 and a second insulation layer that is disposed above the first insulation layer 452. The first insulation layer 452 is disposed in an area that corresponds to the measurement electrodes 420, the measurement lines 430, the test lines 440, and surrounding portions thereof in a plan view. The first insulation layer 452 covers the first line section 444A and the measurement lines 430 from above.

The first test electrode sections 442ₙ, 442ₙ₊₂ . . . of the first test line 440A and the second test line 440B (namely, the second test electrode sections 442ₙ₊₁, 442ₙ₊₃ . . . and the second line section 444B) are disposed on the first insulation layer 452. Furthermore, in this embodiment, the shield layers 446 are additionally disposed on the first insulation layer 452 so as to overlap the respective measurement lines 430. The first line section 444A of the first test line 440A and the first test electrode sections 442ₙ, 442ₙ₊₂ . . . are connected via the contact holes CH that are formed through the first insulation layer 452.

The second insulation layer 454 is disposed on the first insulation layer 452 so as to cover the first test electrode sections 442ₙ, 442ₙ₊₂ . . . , the second test line 440B, and the shield layers 446 from above.

The first measurement electrodes 420 are disposed on the second insulation layer 454. The first measurement electrodes 420 and the measurement lines 430 are connected via the contact holes CH that are formed through the first insulation layer 452 and the second insulation layer 454.

When the test signal is sent to the first test line 440A, the test signal is sent to the first test lines 430ₙ, 430ₙ₊₂ . . . via the first line section 444A, the first test electrode sections 442ₙ, 442ₙ₊₂ . . . , and the first measurement electrodes 420ₙ, 420ₙ₊₂ . . .

When the test signal is sent to the second test line 440B, the test signal is sent to the second test lines 430ₙ₊₁, 430ₙ₊₃ . . . via the second line section 444B, the second test electrode sections 442ₙ₊₁, 442ₙ₊₃ . . . , and the second measurement electrodes 420ₙ₊₁, 420ₙ₊₃ . . .

When the testing is performed with using the first test line 440A, no test signals is sent to the second measurement electrodes 420ₙ₊₁, 420ₙ₊₃ . . . . When the testing is performed with using the second test line 440B, no test signals is sent to the first test electrode sections 442ₙ, 442ₙ₊₂ . . .

The first shield layers 446ₙ, 446ₙ₊₂ . . . are connected to the second measurement electrodes 420ₙ₊₁, 420ₙ₊₃ . . . respectively, such that the second line section 444B can be grounded when the testing is performed with using the first test line 440A. According to such a configuration, when the testing is performed with using the first test line 440A, the first measurement lines 430ₙ, 430ₙ₊₂ . . . can be effectively shielded from noise.

The second shield layers 446ₙ₊₁, 446ₙ₊₃ . . . are connected to the first measurement electrodes 420, 420ₙ₊₂ . . . , respectively, such that the first line section 444A can be grounded when the testing is performed with using the second test line 440B. According to such a configuration, when the testing is performed with using the second test line 440B, the second measurement lines 430ₙ₊₁, 430ₙ₊₃ . . . can be effectively shielded from noise.

In the above embodiment, the measurement electrodes 420 include first measurement electrodes and second measurement electrodes. The measurement lines 430 include first measurement lines that are connected to the first measurement electrodes, respectively, and second measurement lines that are connected to the second measurement electrodes, respectively. The test line 440 includes test electrode sections and line sections. The test electrode sections 442 include first test electrode sections that are opposite the first measurement electrodes, respectively, via the insulation layer 450 and second test electrode sections that are opposite the second measurement electrodes, respectively, via the insulation layer 450. The test line sections include the first line section 444A that is connected to the first test electrode sections and the second line section 444B that is connected to the second test electrode sections. According to such a configuration, in the configuration including a large number of the measurement electrodes 420 and the measurement lines 430 on the base plate 10, the test lines 440 can be easily disposed by defining the test lines 440 in multiple groups (two groups in this embodiment). In performing the testing of the measurement electrodes 420 and the measurement lines 430 with using the measurement lines 430 of one single group, if an error occurs in any one of the measurement lines 430, the testing for all of the measurement electrodes 420 and the measurement lines 430 cannot be performed. On the other hand, with the test lines 440 being defined into multiple groups, such a problem is less likely to be caused.

In the above embodiment, the first measurement electrodes and the second measurement electrodes are arranged alternately along one direction on the surface of the base plate. The insulation layer 450 includes the first insulation layer 452, which is disposed between one of the base plate 10 and the first line section 444A and corresponding one of the first test electrode sections, the second test electrode sections, and the second line section 444B, and the second insulation layer 454, which is disposed above the first test electrode sections, the second test electrode sections, and the second line section 444B and below the first measurement electrodes and the second measurement electrodes. The first line section 444A and the first test electrode sections are connected via through portions that are through the first insulation layer 452. According to such a configuration, the first test line 440A and the second test line 440B can be preferably formed with suppressing occurrence of short-circuits.

In the above embodiment, the first shield layer 446ₙ, 446ₙ₊₂ . . . is disposed above the first measurement line 430ₙ, 430ₙ₊₂ . . . and between the first insulation layer 452 and the second insulation layer 454. The first shield layer 446ₙ, 446ₙ₊₂ . . . is connected to the second test electrode section 442ₙ₊₁, 440ₙ₊₃ . . . that is adjacent to the first measurement line 430ₙ, 430ₙ₊₂ . . . . The second shield layer 446ₙ₊₁, 446ₙ₊₃ . . . is disposed above the second measurement line 430ₙ₊₁, 430ₙ₊₃ . . . and between the first insulation layer 452 and the second insulation layer 454. The second shield layer 446ₙ₊₁, 446ₙ₊₃ . . . is connected to the first test electrode section 442ₙ₊₂, 442ₙ₊₄ . . . that is adjacent to the second measurement line 430ₙ₊₁, 430ₙ₊₃ According to such a configuration, the first test electrode section is continuous to the second shield layer and the second test electrode section is continuous to the first shield layer. Therefore, the test electrode sections and the shield layers can be preferably formed with lithography. With using the test line 440, the first shield layer and the second shield layer can be grounded at any appropriate timing. Therefore, testing of electrically conductive state and measuring of action potentials can be performed quite precisely.

### Sixth Embodiment

A sixth embodiment will be described with reference to FIG. 15. In a cell potential measuring device 500 of the sixth embodiment, measurement electrodes 520 (sixteen measurement electrodes in the drawing) are arranged in rows and columns (for instance, four rows and four columns) in a center portion of a square base plate 510. Although details are not illustrated, a measurement line 530 and a test line 540 (namely, a test electrode section and a line section) are disposed on the base plate 10 for each measurement electrode 520. An insulation layer (not illustrated) is provided for appropriately insulating them. Configurations, operations, and effects of the measurement electrodes 520, the measurement lines 530, and the test lines 540 are similar to any one of the first to fifth embodiments and will not be described.

The measurement lines 530 extend to each of two side edges of the base plate 510. Connection terminal groups 538A, 538B are disposed on the two side edges of the base plate 510, respectively. Ends of the measurement lines 530 are connected to each of the connection terminal groups 538A, 538B. The test lines 540 extend to each of another two side edges of the base plate 510. Connection terminal groups 548A, 548B are disposed on the other two side edges of the base plate 510, respectively. Ends of the test lines 540 are connected to each of the connection terminal groups 548A, 548B.

A wall 560 having a ring shape is disposed on the base plate 510 so as to surround all the measurement electrodes 520. The cell culture environment is created inside the wall 560. A configuration of the wall 560 is not particularly limited. The wall 560 is preferably made of the above-described transparent material having biocompatibility so as to be appropriate for culturing and observing cells. The wall 560 is disposed on an upper surface of the base plate 510 or on an insulation layer 550, which covers the lines, to be watertight via adhesive, for example.

With the above configuration, the cell potential measuring device 500 can preferably measure action potentials with performing the cell culture. The cell potential measuring device 500 can preferably test the electrically conductive state between each of the measurement electrodes 520 and corresponding one of the measurement lines 530 at preferable timing with performing the cell culture.

### Seventh Embodiment

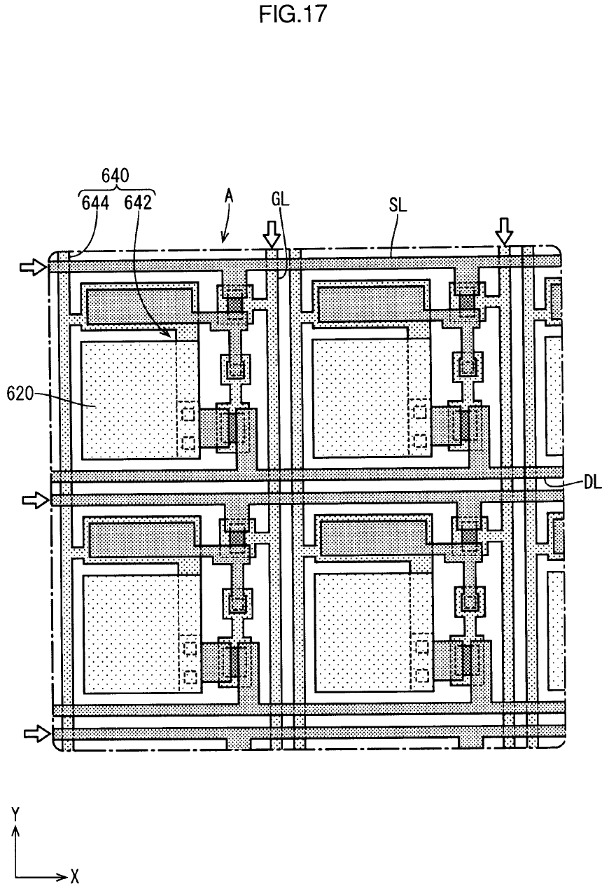

A seventh embodiment will be described with reference to FIGS. 16 to 21. X and Y in the drawings represent a row direction and a column direction along the surface of a base plate 610, respectively, and the row direction and the column direction are perpendicular to each other. The directions are temporally specified and may not be interpreted in a limited way. A symbol is applied to one of the same components and may not be applied to other components.

In the cell potential measuring device 500 of the sixth embodiment, the measurement electrodes 520 (sixteen measurement electrodes, for example, and a hundred or less typically) are arranged on the square base plate 510. As illustrated in FIG. 16, in a cell potential measuring device 600 of the seventh embodiment, multiple (typically, a hundred or more, and for instance, two hundreds or more, and five hundreds or more) measurement electrodes 620 are disposed on a square base plate 610. The number of measurement electrodes 620 disposed on the base plate 610 is determined with considering a size of the base plate (namely, a size of a cell culture area), a size of the measurement electrode 620, and a distance (an interval) between adjacent measurement electrodes 620. Although details are not illustrated, the measurement electrodes 620 are arranged in rows and columns (a hundred rows and a hundred columns, for example) in a center portion of the base plate 610. Other than the following description, this embodiment is similar to any of the first to sixth embodiments and the similarities will not be described.

FIG. 17 is an enlarged view illustrating an area of the cell potential measuring device 600 in which the four measurement electrodes 620 are arranged. On the base plate 610, line sections 644 of test lines 640 and signal lines GL, which extend in the column direction, are disposed. Furthermore, on the base plate 610, source lines SL and detection lines DL, which extend in the row direction, are disposed. An area that is surrounded by the four lines 644, GL, SL, DL is defined as a measurement area A. The measurement electrode 620 and a test electrode section 642 that is opposite the measurement electrode 620 are arranged in one measurement area A.

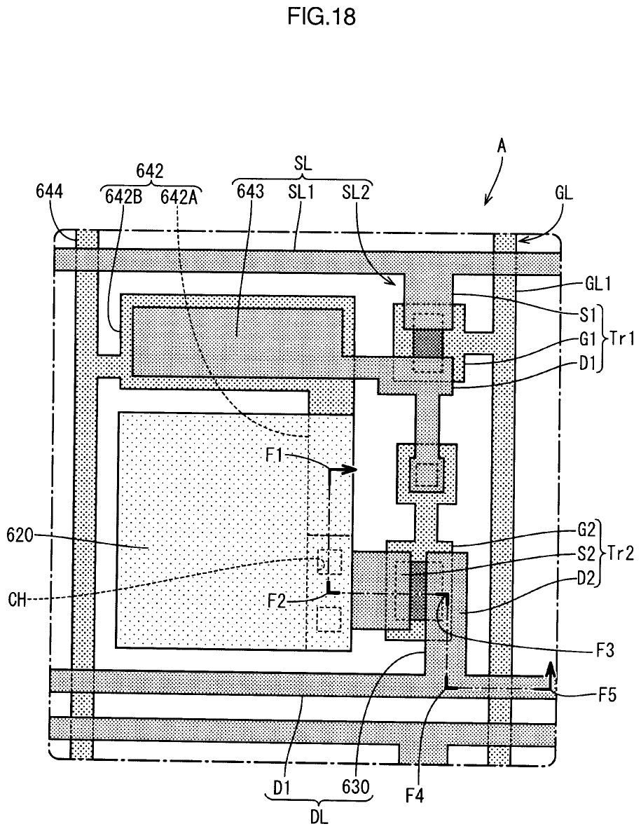

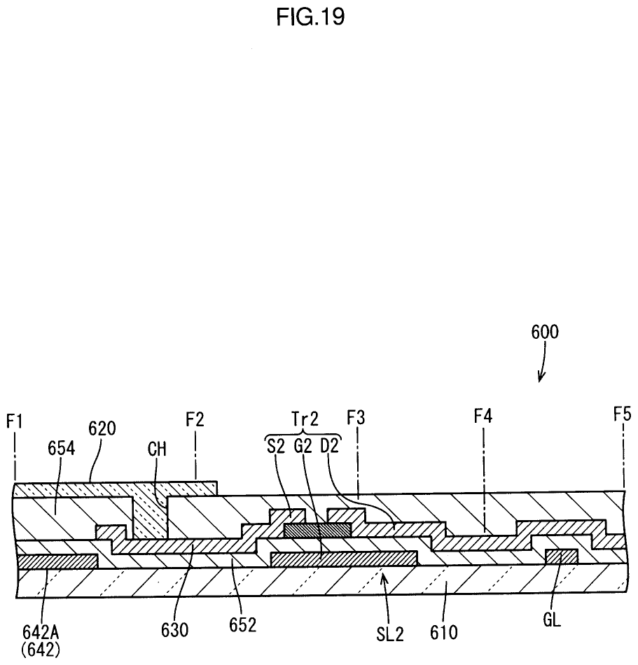

As illustrated in FIGS. 18 and 19, the line section 644 is connected to multiple test electrode sections 642. The test electrode section 642 of this embodiment includes an opposing portion 642A and a capacitance portion 642B. The opposing portion 642A is opposite the measurement electrode 620 via insulation layers 652, 654 and they are configured as a capacitor. The capacitance portion 642B is a charge storing portion that can supply a large amount of charges promptly to the opposing portion 642A. The capacitance portion 642B is disposed closer to the line section 644 than the opposing portion 642A is. The capacitance portion 642B is not opposite the measurement electrode 620.

A portion of the measurement electrode 620 is opposite the opposing portion 642A and other portion of the measurement electrode 620 is not opposite the opposing portion 642A. Although a configuration is not limited thereto, a non-opposing portion, which is not opposite the opposing portion 642A (the test electrode section 642), is a large area in the measurement electrode 620. Therefore, no insulation layers 652, 654 are disposed in the non-opposing portion. The insulation layers 652, 654 are disposed in the portions where the lines 644, GL, SL, DL need to be insulated from the outside. The non-opposing portion of the measurement electrode 620 is disposed directly on the base plate 610. In this embodiment, an area of the non-opposing portion (that is substantially equal to a contact area of the measurement electrode 620 and the base plate 610) of the measurement electrode 620 is 50% or more (for instance, 80% or more) of the area of the measurement electrode 620. The area of the non-opposing portion is 30% or more (for instance, 50% or more) of an area of the measurement area A.

Test signals are sent via the signal lines GL to the test electrode sections 642 in cooperation with the line sections 644 and the source lines SL. The source line SL will be described. The source line SL includes a main line section SL1, capacitance electrode sections 643, and switch line sections SL2. The main line section SL1 extends in the row direction. One capacitance electrode section 643 and one switch line section SL2 are arranged in every measurement area A.

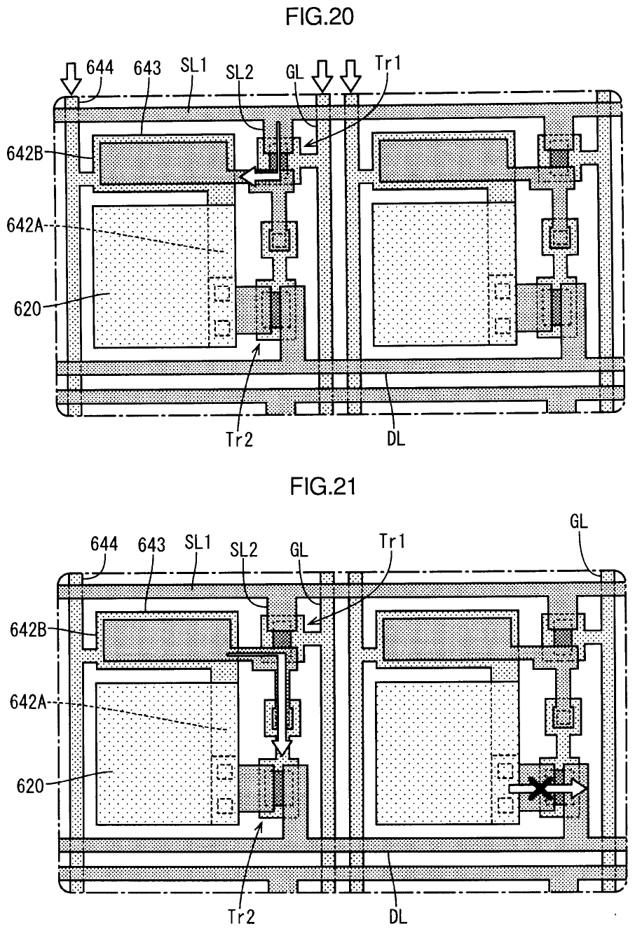

The switch line section SL2 includes a first switching element Tr1 and is connected to a second switching element Tr2. Each of the first switching element Tr1 and the second switching element Tr2 includes a thin film transistor TFT (one example of a field effect transistor). More specifically, the switch line section SL2 connects the main line section SL1 and the capacitance electrode section 643 and is a component for activating the second switching element Tr2 of a measurement line 630, which will be described later. The switch line section SL2 includes the first switching element Tr1 between the main line section SL1 and the capacitance electrode section 643. A source S1 of the first switching element Tr1 is connected to the main line section SL1. A drain D1 of the first switching element Tr1 is connected to the capacitance electrode section 643 and a gate G2 of the second switching element Tr2. A gate G1 of the first switching element Tr1 is connected to the signal line GL. As illustrated in FIG. 20, with a driving signal being sent from the signal line GL to the first switching element Tr1, the source S1 and the drain D1 of the first switching element Tr1 are electrically connected and charges are transferred from the source line SL to the capacitance electrode section 643.

The capacitance electrode section 643 is an element for storing charges in the capacitance portion 642B of the test electrode section 642. The capacitance electrode section 643 is disposed opposite the capacitance portion 642B via an insulation layer (not illustrated). The capacitance electrode section 643, the insulation layer, and the capacitance portion 642B are configured as a capacitor. With the capacitance electrode section 643 being supplied with charges, the charges are induced to the capacitance portion 642B via the line section 644. Accordingly, charges are stored in the capacitance portion 642B. With sufficient charges being stored in the capacitance electrode section 643, the potential of the switch line section SL2 is increased. Thus, as illustrated in FIG. 21, the second switching element Tr2 is activated.

The detection line DL includes a main line section DL1 and measurement lines 630. The main line section DL1 extends along the row direction. One measurement line 630 is disposed in every measurement area A. One ends of the measurement lines 630 are connected to the main line section DL1. Other ends of the measurement lines 630 are connected to the measurement electrodes 620, respectively. The measurement line 630 includes the second switching element Tr2. The gate G2 of the second switching element Tr2 is connected to the switch line section SL2, as previously described. The source S2 of the second switching element Tr2 is connected to the measurement electrode 620. The drain D2 of the second switching element Tr2 is connected to the main line section DL1. With sufficient amount of charges being stored in the capacitance electrode section 643, a test signal is transferred to the measurement electrode 620 and the second switching element Tr2 is activated.

With the above configuration, the electrically conductive state of the measurement electrodes 620 and the measurement lines 630 can be tested with the following steps. First, the potential of the main line section SL1 of the source line SL is adjusted such that the sufficient charges for activating the second switching element Tr2 can be stored in the capacitance electrode section 643. Next, a driving signal for driving the first switching element Tr1 is sent via the signal line GL. Accordingly, the first switching element Tr1 of the switch line section SL2 is activated (refer to FIG. 20). As a result, the charges move from the main line section Sll of the source line SL to the capacitance electrode section 643. Then, the charges are stored between the capacitance electrode section 643 and the capacitance portion 642B and the second switching element Tr2 is activated (refer to FIG. 21). After charging, the supplying of driving signals via the signal line GL is stopped and a current flow to the first switching element Tr1 is stopped.

In the above state, the test signal is sent from the test electrode section 642 (the opposing portion 642A) to the measurement electrode 620. The measurement electrode 620 and the measurement line 630 are electrically conductive. Therefore, when the test signal is input to the test electrode section 642 via the line section 644 and the electrically conductive state of the measurement electrode 620 and the measurement line 630 is good, the test signal is sent to the main line section DL1 of the detection line DL via the second switching element Tr2. The output test signal has a waveform corresponding to the electrically conductive state of the measurement electrode 620 and the measurement line 630. Therefore, by analyzing the waveform, the details of the electrically conductive state can be obtained. On the other hand, when the electrically conductive state of the measurement electrode 620 and the measurement line 630 is not good, the test signal is not output to the main line section DL1 of the detection line DL. Accordingly, abnormality in the electrical conductivity can be confirmed.

In the measurement areas A where driving signals and charges are not supplied via the signal lines GL and the source lines SL, the first switching elements Tr1 and the second switching elements Tr2 are not activated and no test signal is sent via the line sections 644. Therefore, among the measurement electrodes 620, target measurement electrodes 620 (the measurement areas A) that are to be tested can be selected.

When the cells generate action potentials, the action potentials can be detected with the following steps. Similar to the testing of the electrically conductive state as described above, charges are stored between the capacitance electrode section 643 and the capacitance portion 642B in desired one of the measurement areas A (the measurement electrodes 620) to activate the second switching element Tr2 (ON state). When the cell generates an action potential in this state, the action potential propagates to the measurement electrode 620. As a result, the action potential signal received by the measurement electrode 620 is sent to the main line section DL1 of the detection line DL via the second switching element Tr2. Thus, the potential of the cell can be measured with high accuracy with using the electrode whose electrically conductive state has been tested.

The wall 660 having a ring shape projects from the base plate 610 and extends to surround the measurement electrodes 620. The connection terminals 662 are disposed on the edge portions of the base plate 610. The line sections 644, the signal lines GLG, the source lines SL, and the detection lines DL extend to the edge portions of the base plate 610 and are connected to the connection terminals 662. The driving signals for driving the first switching element Tr1 and the test signals are input via the connection terminals 662. Transmission of the driving signals for the first switching element Tr1, transmission of the test signals, and analysis of the output test signals can be performed with using the known testing technology of testing liquid crystal panels.

### Other Embodiments

The technology described herein is not limited to the embodiments described above and illustrated by the drawings. For example, the following embodiments will be included in the technical scope of the present technology.

(1) In the above embodiments, the base plate 10 is a transparent glass plate having no color. However, the base plate 10 does not necessarily have this configuration. For instance, the base plate may be made of white or black material when acting potentials of cells are measured with chemiluminescence or fluorescence.

In the above embodiments, the cell potential measuring device includes the base plate, the measurement electrodes, the measurement lines, and the test lines. The cell potential measuring device may include a structure layer other than the measurement electrodes, the measurement lines, and the test lines as long as the subject matter of the present technology can be maintained. An example of such a structure layer is a protection layer.

(3) In the first to fourth embodiments, the insulation layer is disposed in an area where the measurement electrodes are arranged and the surrounding portions thereof in a plan view. However, the insulation layer may not be disposed between the measurement electrodes and the base plate in areas where the measurement electrodes can be surely insulated from the test line section.

(4) In the above embodiments, the cell potential measuring device that includes thin film transistors may be produced with appropriately using the known TFT array producing technology.

(5) In the above embodiments, the cell potential measuring device includes the base plate, the measurement electrodes, the measurement lines, and the test lines. The cell potential measuring device may additionally include a processing device that processes signals related to the action potentials obtained via the electrodes and a display that displays analysis results. The processing device may include a microcomputer, for instance. The processing device may be configured to execute an analysis program for analyzing the signal data obtained from the electrode to count nerve actions (spikes), detect burst, and analyze a network of the cells in measuring action potentials for a long time. With respect to myocytes such as myocardium, outside potentials may be measured or response potential data related to various kinds of reactions in response to contraction and relaxation of myocardium may be analyzed by the processing device.

### EXPLANATION OF SYMBOLS

1, 100, 200, 300, 400, 500, 600 . . . cell potential measuring device, 10, 510, 610 . . . base plate, 10A . . . upper surface, 20, 320, 420, 520, 620 . . . measurement electrode (first electrode), 20A . . . upper surface, 30, 330, 430, 530, 630 . . . measurement line (first line), 38, 48 . . . connection terminal, 40, 140, 240, 340, 440, 540, 640 . . . test line (second line), 42, 142, 242, 342, 442, 642 . . . test electrode section (second elect rode section), 442n . . . first test electrode section (third electrode section), 442n . . . second test electrode section (fourth electrode section), 44, 144, 444, 644 . . . line section, 346, 446 . . . shield layer, 50, 150, 250, 450, 550 . . . insulation layer, 152, 252, 352, 452 . . . first insulation layer, 154, 254, 354, 454 . . . second insulation layer, 560, 660 . . . wall, 662 . . . connection terminal

1. A cell potential measuring device comprising:
a base plate having insulation properties;
a first line disposed on the base plate;
an insulation layer disposed on the base plate and covering at least a surface of the first line with a through hole;
a first electrode disposed on the insulation layer and being electrically connected to the first line via the trough hole; and
a second line disposed on the base plate and a portion of which is disposed below the first electrode via the insulation layer.

2. The cell potential measuring device according to claim 1, wherein
the second line includes
a second electrode section that is opposite the first electrode, and
a line section that extends from the second electrode section, and
a distance between the first electrode and the second electrode section is 10 nm or more and 100 μm or less.

3. The cell potential measuring device according to claim 1, wherein the insulation layer covers a surface of the second line.

4. The cell potential measuring device according to claim 1, wherein the second line is disposed directly on the base plate.

5. The cell potential measuring device according to claim 1, wherein the insulation layer includes
a first insulation layer that is disposed between the base plate and the second line, and
a second insulation layer that is disposed between the second line and the first electrode.

6. The cell potential measuring device according to claim 1, wherein
the second line includes
a second electrode section that is opposite the first electrode, and
a line section that extends from the second electrode section, and
the line section is disposed directly on the base plate,
the insulation layer includes
a first insulation layer that is disposed between the base plate and the second electrode section, and
a second insulation layer that is disposed between the second electrode section and the first electrode.

7. The cell potential measuring device according to claim 6, wherein the line section and the second electrode section are made of different materials.

8. The cell potential measuring device according to claim 1, wherein the first electrode includes transparent electrically conductive material that includes at least one kind selected from the group consisting of tin oxide, zinc oxide, indium zinc oxide, and indium tin oxide.

9. The cell potential measuring device according to claim 1, wherein at least a portion of the second line and the first line include at least one kind selected from the group consisting of gold, silver, copper, aluminum, tantalum, tungsten, molybdenum, niobium, and titanium.

10. The cell potential measuring device according to claim 1, wherein
the insulation layer includes
a first insulation layer at least a portion of which is disposed directly on the base plate, and
a second insulation layer at least a portion of which is disposed directly under the first electrode,
each of the first insulation layer and the second insulation layer includes an overlapping portion that is disposed above the first line, and
a shield layer having electrical conductivity is disposed between the overlapping portion of the first insulation layer and the overlapping portion of the second insulation layer.

11. The cell potential measuring device according to claim 1, wherein
the first electrode includes first electrodes one of which is defined as a third electrode and another one of which is defined as a fourth electrode,
the second line includes a second electrode sections that are opposite the first electrodes and a line section that extends from the second electrode sections,
one of the second electrode sections is defined as a third electrode section that is opposite the third electrode and another one of the second electrode sections is defined as a fourth electrode section that is opposite the fourth electrode, and
the third electrode section and the fourth electrode section are connected to the line section.

12. The cell potential measuring device according to claim 1, wherein
the first electrode includes first electrodes, some of the first electrodes are defined as third electrodes and other ones of the first electrodes are defined as fourth electrodes,
the first line includes first lines, some of the first lines are defined as third lines that are connected to the third electrodes, respectively, and other ones of the first lines are defined as fourth lines that are connected to the fourth electrodes, respectively,
the second line includes one second lines and another second line, and each of the second lines includes second electrode sections and a line section,
the one second line includes the second electrode sections defined as third electrode sections that are opposite the third electrodes, respectively, and the line section defined as a first line section that is connected to the third electrode sections, and
the other second line includes the second electrode sections are defined as fourth electrode sections that are opposite the fourth electrodes, respectively, and
the line section defined as a second line section that is connected to the fourth electrode sections.

13. The cell potential measuring device according to claim 12, wherein
the third electrodes and the fourth electrodes are arranged alternately along one direction along a surface of the base plate,
the insulation layer includes
a first insulation layer that is disposed above the base plate and the second first line section and below a corresponding one of the third electrode sections, the fourth electrode sections, and the first line section, and
a second insulation layer that is disposed above the third electrode sections, the fourth electrode sections, and the first line section and below the third electrodes and the fourth electrodes, and
the second line section and the fourth electrode sections are connected via trough portions that is through the first insulation layer.

14. The cell potential measuring device according to claim 13, further comprising:
first shield layers disposed above the third lines, respectively, and between the first insulation layer and the second insulation layer, the first shield layers being connected to the fourth electrode sections, respectively, that are adjacent to the third lines, respectively; and
second shield layers disposed above the fourth lines, respectively, and between the first insulation layer and the second insulation layer, the second shield layers being connected to the third electrode sections, respectively, that are adjacent to the fourth lines, respectively;

15. The cell potential measuring device according to claim 1, further comprising a wall that projects from the base plate and surrounds the first electrode.

16. The cell potential measuring device according to claim 1, further comprising a field effect transistor that is disposed on the base plate and connected to the second line.

17. The cell potential measuring device according to claim 1, further comprising a field effect transistor that is disposed on the base plate and connected to the first line.
